(19) United States
(12) Patent Application Publication
(10) Pub. No.: US 2012/0245560 A1
(43) Pub. Date: Sep. 27, 2012

(54) MULTI-VESSEL COMPUTER CONTROL DRUG DELIVERY

(76) Inventor: MARK N. HOCHMAN, Great Neck, NY (US)

(21) Appl. No.: 13/053,518

(22) Filed: Mar. 22, 2011

(51) Int. Cl. A61M 5/31

(52) U.S. Cl. 604/518; 604/82

(57) ABSTRACT

Apparatus for combining and subcutaneously injecting a mixture of drug and other liquid such as a buffering solution, uses cartridges with cylinders containing the drug and other liquid, and pistons pushable toward discharge ends of the cylinders for discharging the liquids. A drive unit housing with motor therein is activated by computer to linearly move a motor plunger. A multiple cartridge holder receives cartridges with the drug and other liquid and has a mixing chamber with inlets connected to the discharge ends for receiving a mixture of drug and other liquids. Micro-tubing is connected to an outlet of the chamber and has a handpiece with subcutaneous injection needle at its opposite end for receiving and injecting the mixture. Plural cartridge plungers are engages via a motion transmission to the motor plunger and move into open ends of the cartridges to push the pistons and discharge liquids from the cartridges into the mixing chamber.

### FIELD AND BACKGROUND OF THE INVENTION

The present invention relates in general to the delivery of two or more liquids to a subject through a needle, and in particular, to a new and useful apparatus and method for the simultaneous, computer-controlled, mixing together, proportioning and delivery of a liquid analgesic plus a buffer solution to the subject by subcutaneous injection.

It is known in both the medical and the dental literature to use a mixture of two liquids such as sodium bicarbonate and local anesthetic solution to increase the general effectiveness of the local anesthesia. The major problem has been the need to mix the two agents chair-side, immediately before injection, because of the volatility of the mixed ingredients. It is the buffering agent, sodium bicarbonate, that must be mixed into a second drug chair-side at the time of injection because it is a highly unstable chemical. Therefore, in order to clinically utilize a buffering agent in the dental environment (e.g. sodium bicarbonate mixture with local dental anesthetic solution) a chair-side pre-injection mixing procedure is required. Currently there is no convenient and cost effective way of preparing a sodium bicarbonate-buffered local anesthetic agent.

The use of Sodium Bicarbonate Inj., 8.4% USP Neutralizing Additive Solution has been found beneficial in a 10:1 ratio with lidocoaine/epinephrin liquid analgesic. Ten times by volume of the analgesic plus one time by volume of the sodium bi-carbonate solution are mixed together and are then immediately injected into a patient at the location where pain relief or pain suppression is intended. Such a solution is supplied by Onpharma, Inc. of Los Gatos, Calif. It is warned that once mixed the analgesic plus sodium bicarbonate solution must be immediately injected and not stored. It is also emphasized that the solution is clear, colorless and free of particles or cloudiness and this should be verified before use. The known purpose for using sodium bicarbonate with the analgesic is to reduce pain by making the solution closer in pH to the patient's own body tissue, thus buffering the mixture of analgesic and solution. Onpharma, Inc. provides a rather complex and expensive apparatus for manually mixing and injecting the buffered mixture.

Currently there are two general methods of combining buffering agent with liquid analgesic.

In a first general method a clinician mixes local anesthetic (such as lidocaine with epinephrine) chair-side by using a multi-dose vial of sodium bicarbonate. He or she then mechanically draws up the two liquids into a standard medical syringe. The clinician would then have to determine the proper amount of each liquid prior to mixing to ensure that the correct chemical (molar) concentration of sodium bicarbonate was provided so that it would result in the proper proportions of these two substances. This approach is very time consuming and inconvenient. It also presents the risk of contamination of sterile components as they are handled in the clinic.

This first method of chair-side manual mixing of the buffering agent (sodium bicarbonate) with a local anesthetic solution (lidocaine with epinephrine) can also be performed with a dental syringe or dental cartridge system. It would require the clinician to mechanically express a certain portion of the anesthetic solution contained in a standard 1.8 ml dental cartridge, thereby wasting this drug. Once expressed through the needle connected to a standard syringe, the needle would be placed into a multi-dose vial of buffering agent (sodium bicarbonate) and then drawn back into the dental cartridge to mix the two different solutions. This presents the following problems:

It is difficult to properly discard the local anesthetic drug as it is caustic and has potential effects if directly contacted. A manual chair-side mixing using a dental syringe is not convenient or practical to perform.

It is difficult to accurately and properly mix the two drugs in the precise proportions that are needed to make the buffered local anesthetic mixture effective.

There is a potential for cross contamination of the multi-dosage vial and potential of contamination to the local anesthetic solution to be injected into the patient.

This technique requires a pre-injection mixing procedure to be performed manually by a qualified clinician and cannot be properly delegated to a dental assistant.

The second general method to allow chair-side mixing of a local anesthetic solution (i.e. lidocaine with epinephrine) with a buffering agent (i.e. sodium bicarbonate) has recently become commercially available from Onpharma, Inc. This system utilizes three separate components that are required to be purchased to accomplish this task. It requires a Mixing Pen (OnSet®), a Cartridge Connector (OnSet®), and Sodium Bicarbonate Inj., 8.4% USP (Onpharm™), provided in a standard 1.7 ml dental glass cartridge.

The deficiencies of this method include cost and the need for manual preparation and use.

This second method requires the purchase of Mixing Pen (approx. $299.00), Cartridge Connectors (Approx. $47.00/box of 4 connectors), and a Sodium Bicarbonate 1.7 ml cartridge (approx. $50.00 each). Recent discussions with Onpharma sales representatives indicate that subsequent to the initial cost of the Mixing Pen, each mixed cartridge injection will cost an additional $6.47. All costs are approximate, and represent costs additional to amortizing the cost of the Mixing Pen.

This is also a manual chair-side mixing system that introduces the ominous possibility for medical error. The pre-injection mixing procedure is a potential source of operator error of mixing two agents and can result in tissue damage, pain and suffering of the patient. There is a national healthcare agenda underway to reduce potential medical-errors in both medicine and dentistry. Introducing procedures that increase this risk are seen as an unwelcome approach to solving a chair-side clinical technique in the healthcare workplace.

A manual pre-injection mixing procedure represents an inconvenience to the clinician as the procedure cannot be legally delegated to a dental assistant and represents the compounding of a new drug and therefore can only be performed by the attending dentist, physician or pharmacist.

The assignee of the subject application owns several U.S. patents for computer-controlled equipment and methods for automating the injection of subjects with analgesic liquids that have been shown to reduce pain and to make certain specialize inductions, such as injection into the periodontal ligament (PDL), practical. The more material of these patents to the subject invention are listed below and are all incorporated here by reference for their teaching of computer-controlled devices, and peripheral accessories and equipment therefore, that are capable of automating part or all of an injection process.

| Number | Title |
|---|---|
| 7,740,612 | Self-administration injection system. |
| 7,625,354 | Handpiece for fluid administration apparatus. |
| 7,618,409 | Computer controlled drug delivery system . . . |
| 7,449,008 | Drug infusion device with tissue identification . . . |
| 6,966,899 | Hand-piece for injection device . . . |
| 6,945,954 | Drug delivery system with profiles. |
| 6,905,482 | Safety IV catheter infusion device. |
| 6,887,216 | Pressure/force computer controlled drug delivery . . . |
| 6,786,885 | Pressure/force computer controlled drug delivery . . . |
| 6,726,658 | Safety IV catheter infusion device. |
| 6,652,482 | Dental anesthetic and delivery injection unit . . . |
| 6,428,517 | Hand-piece for injection device . . . |
| 6,200,289 | Pressure/force computer controlled drug delivery . . . |
| 6,152,734 | Dental anesthetic and delivery injection unit. |
| 6,132,414 | Cartridge holder for an injection device. |
| 6,022,337 | Dental anesthetic and delivery injection unit. |

A need exists for a system and method that is capable of quickly and automatically proportioning, mixing and injecting two active liquids such as liquid analgesic and buffering solution.

### SUMMARY OF THE INVENTION

It is an object of the present invention to provide an apparatus and a method for the multi-vessel delivery of liquids, particularly an analgesic or drug and a buffering solution, that is transparent so that the practitioner can verify the clear, colorless, non-cloudy and particle free nature of the mixture before injection, and that importantly, substantially simultaneously proportions and mixes the liquids together, and then virtually immediately delivers the mixture to the subject by injection.

Another object of the present invention is to provide an apparatus and method that includes an apparatus for combining and subcutaneously injecting a mixture of drug and other liquid such as a buffering solution, uses sealed cartridges with cylinders containing the drug and other liquid, and pistons pushable toward discharge ends of the cylinders for discharging the liquids. A drive unit housing with motor therein is activated by computer to linearly move a motor plunger. A multiple cartridge holder receives cartridges with the drug and other liquid and has a mixing chamber with inlets connected to the discharge ends for receiving a mixture of drug and other liquids. Micro-tubing is connected to an outlet of the chamber and has a handpiece with subcutaneous injection needle at its opposite end for receiving and injecting the mixture. Plural cartridge plungers are engaged via a motion transmission to the motor plunger and move into open ends of the cartridges to push the pistons and discharge liquids from the cartridges into the mixing chamber.

A still further object of the invention is to provide an apparatus and method for combining together and for injecting into a subject, a mixture of a liquid drug and at least one other liquid in desired proportions, the liquid drug and the at least one other liquid each being provided in respective cartridges, each cartridge having a cartridge cylinder for containing a quantity of liquid with a sealed discharge end and an opposite open end, a cartridge piston in the cartridge cylinder near the opposite open end when the cartridge is full, the piston being pushable toward the discharge end for discharging liquid when the discharge end is punctured, the apparatus comprising: a drive unit housing; a motor mounted in the drive unit housing; a motor plunger engaged to the motor for lineal movement with respect to the drive unit housing when the motor is activated; a computer connected to the motor for activating the motor to move the motor plunger at selected times and at selected speeds; a multiple cartridge holder connected to the drive unit housing and adapted to receive a plurality of the cartridges with one of the cartridges containing the liquid drug and at least one other of the cartridges containing the other liquid; a mixing chamber operatively connected to the multiple cartridge holder and having a plurality of inlets each connectable to the discharge end of one of the cartridges received in the multiple cartridge holder for unsealing each respective discharge end for receiving liquid from each cartridge, the mixing chamber mixing liquids from the cartridges together and having an outlet for discharging a mixture of the liquids; a length of micro-tubing having one end connected to the outlet of the mixing chamber and an opposite end; a handpiece assembly connected to the opposite end of the micro-tubing for receiving the mixture of liquid, the handpiece assembly having a subcutaneous injection needle for injecting the mixture of liquids into a subject; a plurality of cartridge plungers mounted for movement with respect to the drive unit housing and into respective opposite open ends of cartridges received by the multiple cartridge holder for pushing the respective cartridge pistons toward the respective discharge ends to discharge liquids from the cartridges into the mixing chamber; and a motion transmission engaged between the motor plunger and the cartridge plungers for moving the cartridge plungers into the respective opposite open ends of the cartridges when the computer activates the motor to move the motor plunger.

Another object of the invention is to use a foot controller connected to a computer for activating a motor of the drive unit via a program on the computer for combining, proportioning, mixing and injecting of the mixture in a hands-free manner unlike the prior art that requires substantial manual manipulation to perform these tasks.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic perspective view of a first embodiment of the invention;

FIG. 2 is an enlarged perspective view of a multiple cartridge holder of the invention;

FIG. 3 is a schematic elevational view of a second embodiment of the invention;

FIG. 4 is a schematic elevational view of a third embodiment of the invention;

FIG. 5 is a schematic plan view of connectors for a fourth embodiment of the invention;

FIG. 6 is a schematic elevational view of a fourth embodiment of the invention;

FIG. 7 is a schematic elevational view of a fifth embodiment of the invention; and

FIG. 8 is a schematic elevational view of a sixth embodiment of the invention.

### DESCRIPTION OF THE PREFERRED EMBODIMENTS

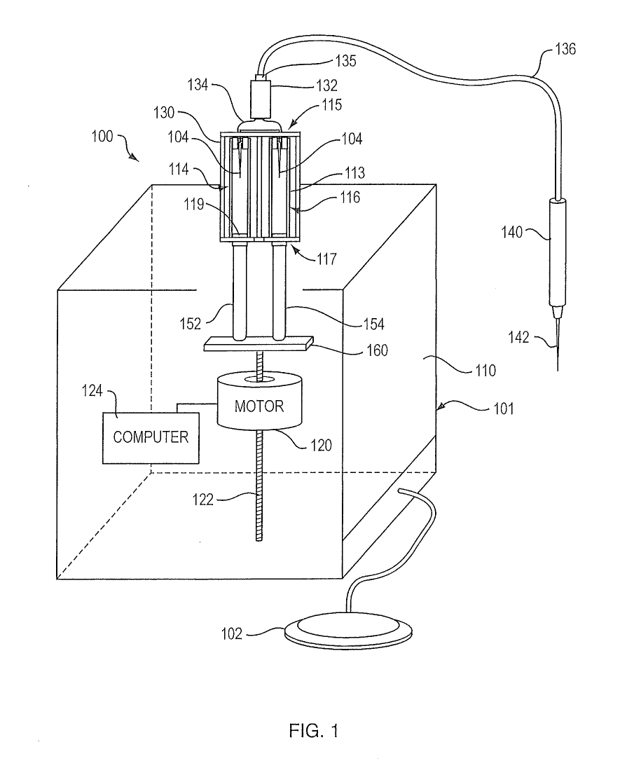

Referring now to the drawings, in which like reference numerals are used to refer to the same or functionally similar elements, FIG. 1 shows an apparatus 100 for combining together and for injecting into a subject or patient, a mixture of a liquid drug such as liquid analgesic, and one or more other liquids such a buffering solution, in desired proportions. The liquid drug and the at least one other liquid are each provided in respective sealed cartridges 114 and 116. These cartridges may be of the type that is known, for example, from U.S. Pat. No. 6,132,414, and each have a transparent cartridge cylinder 113 for containing a quantity of the liquid, with a sealed discharge end 115 that is sealing, for example, by a needle-puncturable rubber stopper, and an opposite open end 117. A cartridge piston 119, e.g. of rubber, is situated inside the cartridge cylinder 113 near the opposite open end 117 when the cartridge is full and the piston 119 is pushable toward the discharge end 115 for discharging the liquid from the cylinder 113, after the discharge end 115 has been punctured, e.g. by a hollow needle 104.

The apparatus 100 comprises a drive unit 101 that is similar to the drive unit of U.S. Pat. No. 6,132,414 but has been adapted for use with the present invention. Drive unit 101 has a drive unit housing 110, a motor 120 mounted in the drive unit housing, and a motor plunger 122 engaged to the motor for lineal movement with respect to the motor and drive unit housing when the motor is activated. For example the motor plunger 122 may be a screw and the motor may include a nut that is rotated when the motor 120 is activated to thereby cause the motor plunger 122 to move upwardly or downwardly in the drive unit housing.

A computer 124 in the drive unit housing 110 is connected to the motor 120 and is programmed to activate the motor to move the motor plunger 122 at selected times and at selected speeds. Examples of this type of motor, plunger and computer control can be found in U.S. Pat. Nos. 6,132,414; 7,740,612; 7,618,409; and 7,449,008.

An important feature of at least one embodiment of the invention is a foot control operated switch that is connected to the bottom of the drive unit. This foot operated switch or foot controller or pedal is an enabling feature that allows mixing to be performed while the operator can simultaneously hand hold the handpiece assembly and needle for insertion into patient. The invention uses this foot controller connected to the computer for activating the motor of the drive unit via the program on the computer for combining, proportioning, mixing and injecting of the mixture in a hands-free manner unlike the prior art that requires substantial manual manipulation to perform these tasks.

Another important feature of at least one embodiment of the invention is a multiple cartridge holder 130 made, for example, of transparent plastic, and detachably connected to the top of drive unit housing 110, over the motor and plunger 120, 122. Holder 130 is adapted to receive a plurality (e.g. two in FIG. 1) of the cartridges 114 and 116, with one of the cartridges, 114 for example, containing the liquid drug and the other of the cartridges (116) containing the other liquid, e.g. a buffering solution. A mixing chamber 132 that is either made as part of the holder 130, or is a separate piece, is in any case operatively connected to the multiple cartridge holder 130 and has a plurality of inlets 134, each connectable, e.g. via needle 104, to the discharge end of one of the cartridges received in the multiple cartridge holder. The respective needles unseal each respective discharge end 115 for receiving liquid from each cartridge. The mixing chamber 132 effects mixing together of the liquids from the cartridges and has an outlet 135 for discharging the mixture of the liquids.

A length of micro-tubing or micro-bore tubing 136, such as that disclosed in U.S. Pat. Nos. 6,132,414 and 7,618,409 for example, has one end connected to the outlet 135 of the mixing chamber 132, and an opposite end connected to a handpiece assembly 140 of the type that is also disclosed in U.S. Pat. Nos. 6,132,414 and 7,618,409 for example, for receiving the mixture of liquid and for injecting it into a subject using a subcutaneous injection needle 142 that is part of the handpiece assembly 140.

For the purpose of the subject invention a subcutaneous injection needle is the type of needle that is commonly used for subcutaneous injections, for example a needle with gauge ranging from 32 gauge to 18 gauge and typically about 25 gauge, and lengths of ½ inch to 1¾ inches.

Inside drive unit 101 an important modification of the known drive units has been made as part of the invention, namely a plurality of cartridge plungers 152 and 154 have been mounted for movement with respect to the drive unit housing 110 and into respective opposite open ends 117 of cartridges 114 and 116 that have been received by the multiple cartridge holder 130. Cartridge plungers 152, 154 are for pushing the respective cartridge pistons 119 toward the respective discharge ends 115 to discharge liquids from the cartridges into the mixing chamber 130. To achieve this, the invention also includes a motion transmission engaged between the motor plunger 122 and the cartridge plungers 152, 154 for moving the cartridge plungers into the respective opposite open ends of the cartridges when the computer 124 activates the motor 120 to move the motor plunger 122. In the embodiment of FIG. 1, the motion transmission is simply a plate 160 for moving the cartridge plungers 152, 154, in the same direction and by the same amount, when motor plunger 122 moves linearly.

Activation of the motor 120 via computer 124 and its programming can be started by a foot controller or pedal 102 that is operatively connected to the computer in a manner that is known, for example, from U.S. Pat. Nos. 6,132,414 and 7,618,409. In this way the combining, proportioning, mixing and injecting of the mixture can be achieved in a hands-free manner.

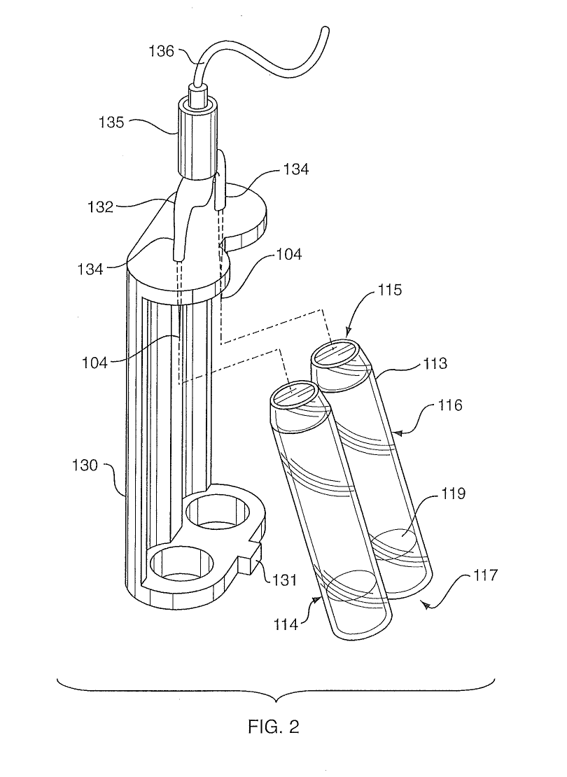

FIG. 2 is an enlarged perspective view of an embodiment of the multiple cartridge holder 130 of the invention made of one or more pieces of strong transparent plastic like clear polycarbonate and having two recessed bays with open fronts for receiving two cartridges 114 and 116. Each cartridge is manipulated in the direction of the curved arrows so as to have its sealed discharge ends 115 punctured by the respective holder needle 104, and its bottom open end 117 pushed into the bottom of its recess to be centered and over an opening in the floor of the holder 130. Once both cartridges are installed in the holder 130, the bottom end of the holder is placed into a correspondingly shaped female pocket in the top surface of the drive unit drive unit housing as shown in FIG. 1, with tabs 131 projecting from the holder floor, lining up with notches at the sides of the pocket to help center and retain the holder 130 to the drive unit housing 110. Latches may also be provided in the housing pocket to engage the tabs or the holder 130 can be shifted to one side to move the tabs 131 under the wall thickness of the housing 110. This shifting will also align the openings in bottom of holder 130 under which are the pistons 119 with the plungers 152 and 154. This engagement is further secured by making sure that the top end of each cartridge plunger 152 and 154 that has been moved to protrude in the housing pocket, has been inserted into one of the holes in the holder floor, and is properly aligned with, or even slightly inserted into the respective bottom open end 117 of each respective cartridge.

This represents a synergistic combination of the known Milestone Scientific injection systems and the new multi-drug delivery system of the present invention.

Before describing additional embodiments of the invention the following comments are offered to more fully explain the use, and the usefulness of the invention.

The invention can be thought of as is a Multi-Cartridge Computer-Controlled Drug Delivery (MCD) Injection Instrument with a unique pre-formed multi-cartridge holder that will create a means for chair-side, fully automated mixing of two or more separate drugs which occurs while simultaneously performing the injection. This is a new chair-side approach to mixing two or more drugs to perform a subcutaneous injection. It eliminates the need for a Pre-Injection Mixing Procedure (PIMP) by allowing two separate pre-filled vessels to be attached or inserted into the cartridge-holder at the time of performing an injection.

The instrument has the following elements:

1) MCD-Drive Unit 101.

2) MCD-Disposable or re-usable Multi-Cartridge Holder 130 with mixing element attached to a micro-tubing and handpiece/subcutaneous injection needle assembly.

3) Unique or Standard Second-Drug Cartridge Container (i.e. Buffering Agent) that has either a standard 1.8 ml glass cartridge configuration. Alternatively the second-drug cartridge can be an uniquely designed configuration which has a “lock-and-key” fit with to the MCD instrumentation, it being understood that the cartridge container is not limited to the 1.8 ml glass dental cartridge. Others fluid volumes, sizes and configurations are anticipated by this disclosure and the design herein discussed.

It is understood that the chemistry and formulation of the Second-Drug-Container can be formulated in such as way that mixing of specific sizes will produced the desired Mixed Formulation of two or more drugs.

The following examples serve only to demonstrate how the invention can be implemented.

Equal mixture of two different drugs utilizing equal volumes.

Utilizing a 1.8 ml glass dental cartridge that is fully filled:

One example is mixing a buffering agent such as Sodium Bicarbonate Inj., 8.4% USP Neutralizing Additive Solution be mixed with Lidocaine with Epinephrine in a ratio that brings the Lidocaine with Epinephrine sufficiently toward the physiologic rang. An alteration of the concentration so that a fully filled 1.8 ml glass cartridge would be formulated to allow the final objective of achieving a mixture of Lidacaine with Epinephrine and a Buffering Agent to have a Neutral pH. This would require the use of a molar concentration when added to the specific volume of 1.8 ml would produce an 8.4% mixture.

The practitioner should choose a volume of Sodium Bicarbonate Inj., 8.4% USP Neutralizing Additive Solution to be mixed with Lidocaine with Epinephrine in a ratio that brings the Lidocaine w/ Epinephrine sufficiently toward the physiologic range to reduce injection pain.

Six clinical study reports have been published in peer-reviewed medical journals between 1987 and 2010, which studies evaluated buffering the pH of Lidocaine w/Epinephrine toward physiologic pH to reduce injection pain by adding 8.4% Sodium Bicarbonate solution in a 10:1 ratio of Lidocaine w/ Epinephrine to 8.4% Sodium Bicarbonate solution. These studies included 268 patients. The studies found that raising the pH of Lidocaine w/ Epinephrine toward physiologic pH using the 10:1 ratio resulted in a reduction in injection pain. In five of the studies, the reduction was statistically significant (p=<0.05); in the sixth there was a strong statistical trend (p=0.06) toward reduced injection pain. There were no reported adverse events.

It is also possible that a increase to a specific volume, such as 1.8 ml would require reformulation of the secondary drug in order to produce the desired molar concentration recommended as well as the final ratio of 10:1 which has be proposed above.

Unequal volumes mixed from two or more different drugs utilizing a unique fluid volume, it is quite possible that a standard vessel such as a 1.8 ml cartridge can be used in which either:

A reduction in drug volume is created by a change in the physical dimensions of the container, i.e. the cartridge is not fully filled.

A reduction in drug volume is created by a change in the physical dimensions of the container, i.e. an over-sized rubber stopper is introduced into the container system that allows the rubber stopper to be positioned analogous to a fully filled 1.8 ml glass cartridge but reduces in internal space by increasing the inner dimensions of the rubber stopper to produce a reduced internal fluid volume to the 1.8 ml cartridge or vessel.

A mixture of different volumes can be produced by alternating the rate of delivery of the different cartridges to produce a specified mixture with a desired formulation.

A mixture of different volumes can be produced by alternating the volume of delivery of the different cartridges to produce a specified mixture with a desired formulation.

It is understood from the description of this invention that it is not limited to any single drug and that when two or more drugs are mixed together and used for subcutaneous drug administration it pertains to this disclosure document. For convenience the example of Lidocaine with Epinephrine mixed with Sodium Bicarbonate has been discussed. It is understood that this description is not limited to these two drugs or any other specific drugs and relates to the mixture of more then one drug.

The MCD-Drive Unit Instrument of the invention should have an important role in allowing the automated mixing of two or more drugs loaded into the MCD-cartridge holder.

The MCD-drive unit may or may not be based on the present Milestone C-CLAD instruments and may or may not utilize patented DPS Dynamic Pressure Sensing® technology currently available in the STA SINGLE TOOTH ANESTHESIA SYSTEM® instrument.

The MCD-drive unit may or may not require a unique disposable cartridge holder connected to a disposable handpiece injection product (currently known as THE WAND® or the STA SINGLE TOOTH ANESTHESIA SYSTEM® Instrument and Handpiece available from Milestone Scientific, Inc.).

The MCD-Drive unit of the invention has the following new design features and methods of use.

Automated chair-side mixing of two or more drugs can be accomplished without the need of an additional PIMP (pre-injection mixing procedure) to be performed.

MCD-Drive can be designed with either the following configurations: Single Motor or Multi-motor (Two electromagnetic motors or more).

Single Motor Design

In one embodiment using a single motor with more then 1 piston (anesthetic cartridge plunger) can be utilized. The plungers are moved from a single electromagnetic or any other piston driven source. The single motor source is not limited to an electromagnetic motor.

The motor can drive a dividing plate 160 that controls multiple plungers either individually or simultaneously.

In the preferred embodiment a single motor simultaneously drives two separate cartridge plungers 152, 154 to advance the rubber pistons 119 in the syringe-like cartridge, or two or more separate cartridges. The simultaneous movement of the plungers results in automated mixing during the simultaneous delivery of mixed and united fluids. The separate drugs are mixed in a common mixing chamber connected to the tubing set that ultimately connects to a handpiece and subcutaneous injection needle. The expressed fluid is auto-mixed upon delivery.

Multi-Motor Design

It is possible that the plungers of two motors in housing 110 will be of two different lengths and that they will move at the same or at different speeds derived from a single motor.

In this embodiment more than one motor is designed to move multiple anesthetic cartridge plungers.

Plungers can move at the same or at different rates of linear travel which can correspond to the delivery of the same or different volumes expressed from the multiple drug vessel sources.

It is possible that one plunger will move at a single given time and not at other times.

The MCD-Drive unit can be programmed to function in a variety of different operations to express drugs from multiple sources simultaneously or independently to creating a mixing of different drugs from various sources.

MCD-Disposable element or re-usable Multi-Cartridge Holder with mixing element can be attached to a micro-tubing and handpiece/needle assembly.

A disposable or re-usable vessel adaptor will allow the insertion or connection of separate drug vessels. A common design feature to the Tubing/Handpiece/Vessel Holder assembly is the design feature of a mixing element.

The mixing element can be a chamber, series of channels or the connection port between the individual drug sources.

In the preferred embodiment the mixing element is a chamber that is integrated in the drug vessel holder.

These Mixing Chamber units mix the separate sources and expresses a mixed drug fluid to the tubing set, handpiece/subcutaneous injection needle assembly for the injection into bodily tissues.

Improvements Over Prior Art

Advantages include:

The MCD System allows automated chair-side drug mixing of two or more drug vessels. Automatically mixed multi-sourced drug instrument in combination with a unique mixing handpiece injection assembly. The system includes the use of a unique or standard vessel containing similar or dissimilar drugs.

The MCD System claims greater efficiency as a Pre-Injection Mixing Procedure is eliminated. This reduces the time required to perform the mixing procedure. It reduces the associated risk of medical errors and human errors when manually mixing two drugs together for injection.

The MCD System can provide verification of drugs being used by use of a unique vessel—vessel holder configuration, i.e. unique “lock-n-key” configuration to the vessel and the holder of the vessel.

The MCD System results in cost savings.

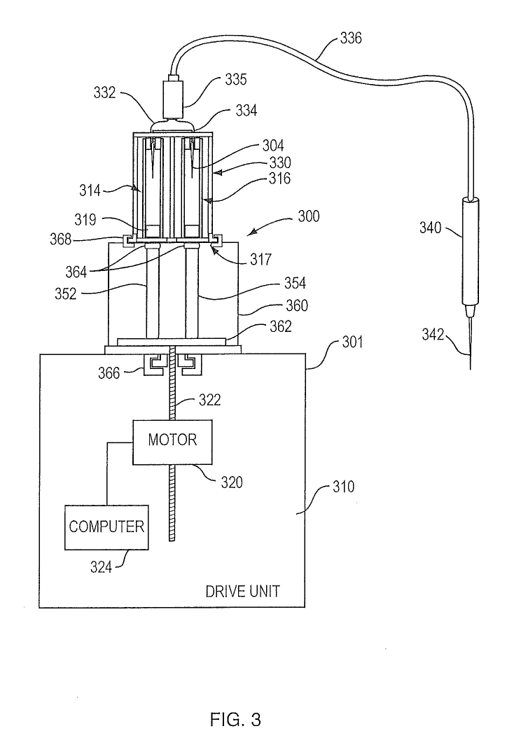

Turning now to the embodiment of FIG. 3, the apparatus 300 of the invention acts to combine together and inject two liquids, preferable two drugs, form respective cartridges 314 and 316. These cartridges may also be of the type known from U.S. Pat. No. 6,132,414, or custom made, but preferably have transparent cartridge cylinders for containing a quantity of the liquid with a sealed discharge end that is a needle-puncturable rubber stopper and an opposite open end 317 with a cartridge piston 319 of rubber inside the cartridge cylinder near the open end when the cartridge is full. The piston 319 is pushable toward the discharge end for discharging the liquid from the cylinder after the discharge end has been punctured, e.g. by a hollow needle 304.

The apparatus 300 comprises a drive unit 301 that can be the same is the drive unit of U.S. Pat. No. 6,132,414 but which is used with an adapter and a multiple cartridge holder 330 of the invention. Drive unit 301 has a drive unit housing 310, a motor 320 mounted in the housing, and a motor plunger 322 engaged to the motor for lineal movement with respect to the motor and housing when the motor is activated. For example the motor plunger 322 may be a screw and the motor may include a nut that is rotated when the motor 320 is activated to thereby cause the motor plunger 322 to move upwardly or downwardly in the housing.

A computer 324 in the housing 310 is connected to the motor 320 and is programmed to activate the motor to move the motor plunger 322 at selected times and at selected speeds. Examples of this type of motor, plunger and computer control can be found again in U.S. Pat. Nos. 6,132,414; 7,740,612; 7,618,409; and 7,449,008.

In this embodiment the multiple cartridge holder 330 made, for example, of transparent plastic, is detachably connected to the top of the adapter 360, and in turn, the adapter is connected to the housing 310, over the motor and plunger 320, 322. Holder 330, which is like the adapter 130 of FIG. 2, receives two cartridges 314 and 316, with one of the cartridges containing the drug and the other containing another drug or other liquid, e.g. a buffering solution. Mixing chamber 332 that is either made as part of the holder 330 or is a separate piece has two inlets 334 each connected e.g. via needle 304, to the discharge end of one of the cartridges. The respective needles unseal each respective discharge end for receiving liquid from each cartridge. The mixing chamber 332 effects mixing together of the liquids from the cartridges and has an outlet 335 for discharging the mixture of the liquids.

A length of micro-tubing or micro-bore tubing 336, such as that disclosed in U.S. Pat. Nos. 6,132,414 and 7,618,409 has one end connected to the outlet 335 of the mixing chamber 332, and an opposite end connected to a handpiece assembly 340 of the type that is also disclosed in U.S. Pat. Nos. 6,132,414 and 7,618,409 for example, for receiving the mixture of liquid and for injecting it into a subject using a subcutaneous injection needle 342 that is part of the handpiece assembly 340.

Adapter 360 is formed of an adapter housing that contains a pusher plate 362 that can be pushed up from the bottom by the motor plunger 322. Two cartridge plungers 352 and 354 are mounted for movement in the adapter housing and to the pusher plate 362. Each cartridge plunger 352, 354 has an upper end with an O-ring 364 that can enter into the into respective opposite open ends 317 of cartridges 314 and 316 that have been received by the multiple cartridge holder 330. Cartridge plungers 352, 354 push the respective cartridge pistons 319 toward the respective discharge ends to discharge liquids from the cartridges into the mixing chamber 330. In this embodiment of the invention, the motion transmission engaged between the motor plunger 322 and the cartridge plungers 352, 354 for moving the cartridge plungers, is the adapter 360 with its pushed plate 362 that moves by the computer 324 activating the motor 320 to move the motor plunger 322.

Key-locks 366 and 368 are used to engage and lock the adaptor housing to the drive unit 301 and the holder 330 to the adaptor 360, respectively. An advantage of this embodiment is that current Milestone Scientific drive units can be used with little or no modifications to inject two or more well mixed and well proportioned drugs to a subject. See U.S. Pat. No. 6,132,414 for details on the type of key-lock that can be used with the present invention.

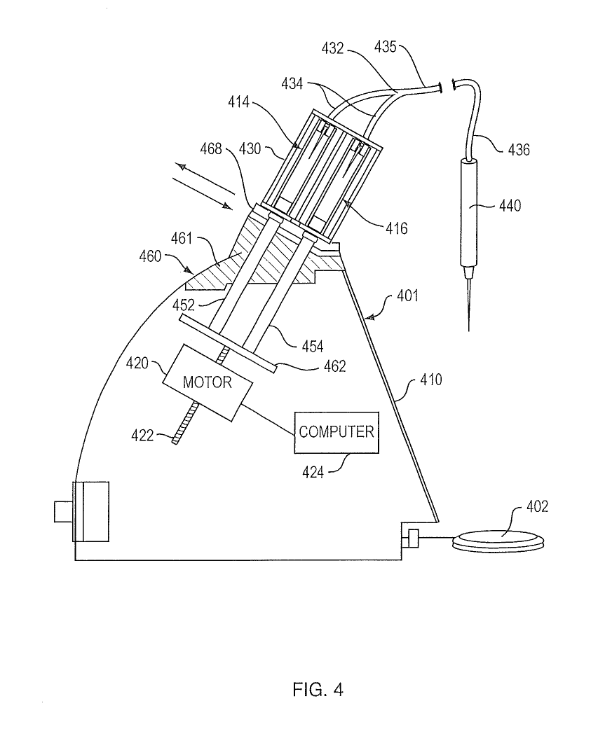

FIG. 4 shows the invention used with a modification to a known Computer Controlled Local Anesthetic Delivery, or C-CLAD instrument system using a drive unit 401 such as these disclosed by U.S. Pat. Nos. 6,132,414 and 7,618,409. This single plunger instrument is modified to accept a dual (or multi) plunger adaptor 460 having a plate 462 on which dual plungers 452 and 454 are directly connected and to which the upper end of the single motor plunger 422 of the single motor 420 of the drive unit 401 is connected. A modified adaptor housing 461 replaces the existing housing cartridge socket of the drive unit of U.S. Pat. No. 7,618,409 or other similar instrument. A dual (or multi) cartridge holder 430 is attached to the adapter housing 461 of the adaptor 460. Holder 430 is attached to the adapter housing 461 by any one of numerous attachment mechanisms 468, such as, but not limited to, sliding in the direction of the arrows, rotating, lock-n-key, etc. Adapter housing 461 is connected to drive unit housing 410. The dual cartridge holder 430 unites two separate sources of drugs in cartridges 414 and 416 via a connector and mixing element attached to a micro-tubing 436 and handpiece/subcutaneous injection needle assembly 440. Computer 424 with its programming and the foot pedal 402 for initialing an injection work n the same was as in the embodiment of FIG. 1.

In the embodiment of FIG. 4, the connector is a Y-shaped section of micro-tubing where the mixing chamber 432 is the junction connecting the two arms 434, 434 of the Y that from the inlets to the mixing chamber 432, and the leg 435 of the Y that forms the mixing chamber outlet that is connected to the micro-tubing 436. Since there will be some length of tubing for the two liquids from cartridges 414 and 416 to travel (at least 6 inches and up to 48 inches) before reaching the handpiece 440, this will allow ample time for full mixing of the liquids before the well-mixed mixture is injected. This represents a synergistic combination of the known micro-tubing of the Milestone Scientific injection systems and the new multi-drug delivery system of the present invention.

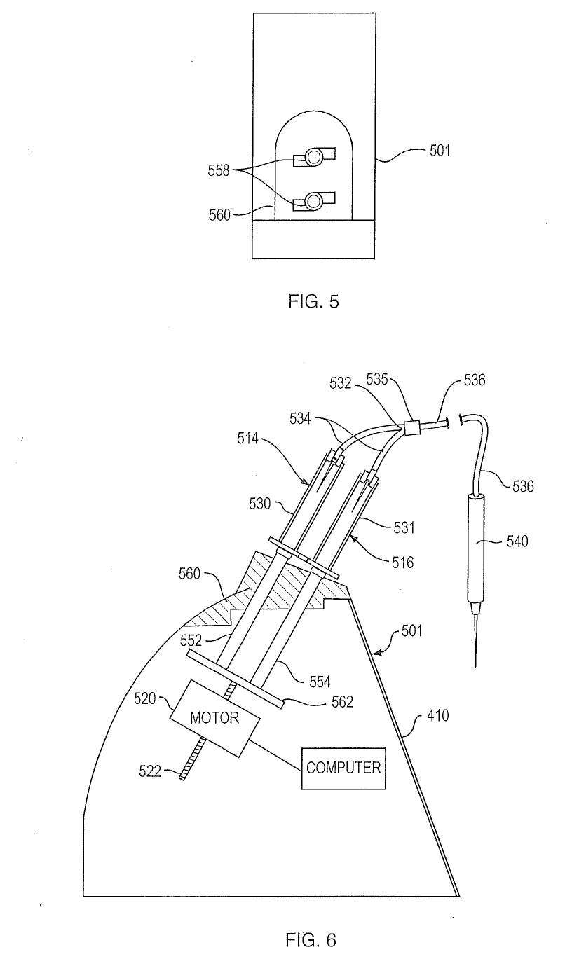

FIGS. 5 and 6 disclose a modification of a standard C-CLAD instrument or drive unit 501, such as, but not limited to, the system of U.S. Pat. No. 7,618,409, in which a single plunger instrument with motor 520 and one motor plunger 522, is refitted to accept a dual plunger adaptor 560 connected to the drive unit housing 510, over the plunger 522, and in which dual plungers 552 and 554 are directly connected to the motor plunger 522 of the drive unit 501 by a plate 562. It is noted that for this disclosure the word “plate” is used to describe any mechanical connection between the end of the motor plunger, and the two or more cartridge plungers.

This embodiment differs from the embodiment of FIG. 4 by accepting two separate cartridge holders 530 and 531 that are independent from one another and that each receive one cartridge 514 and 516 respectively. The separate cartridge holders are united via a tubing connector 532, such as a Y-shaped tubing connector. Connector 532 contains the mixing chamber and communicates with its arms 534 with the discharge ends of the cartridges, and with its leg 535, with the micro-tubing 536 and the handpiece 540. The invention is not limited to a “Y” connector, however, as the only means of uniting two or more separate fluid sources, as any means of uniting two independent tubing or cartridge holders is acceptable. This particular design is ability to use a single standard cartridge holder without a secondary drug fluid source. Hence, this multi-plunger housing adaptor can either be used for multi-cartridge fluid administration or as a single cartridge fluid administration.

Each cartridge holder 530 and 531 has a male end with radial tabs that fit into one of the female keyholes 468 on the adaptor 560 as best shown in FIG. 5, and is turned a quarter turn to lock the cartridge holder to the drive unit in the same way the locking system works in U.S. Pat. No. 7,625,354 for a Handpiece for Fluid Administration Apparatus.

The features of the embodiments of FIGS. 1 to 6 can be used in combination with one another. Any embodiment can be used as a single cartridge administration or a multi-cartridge drug administration.

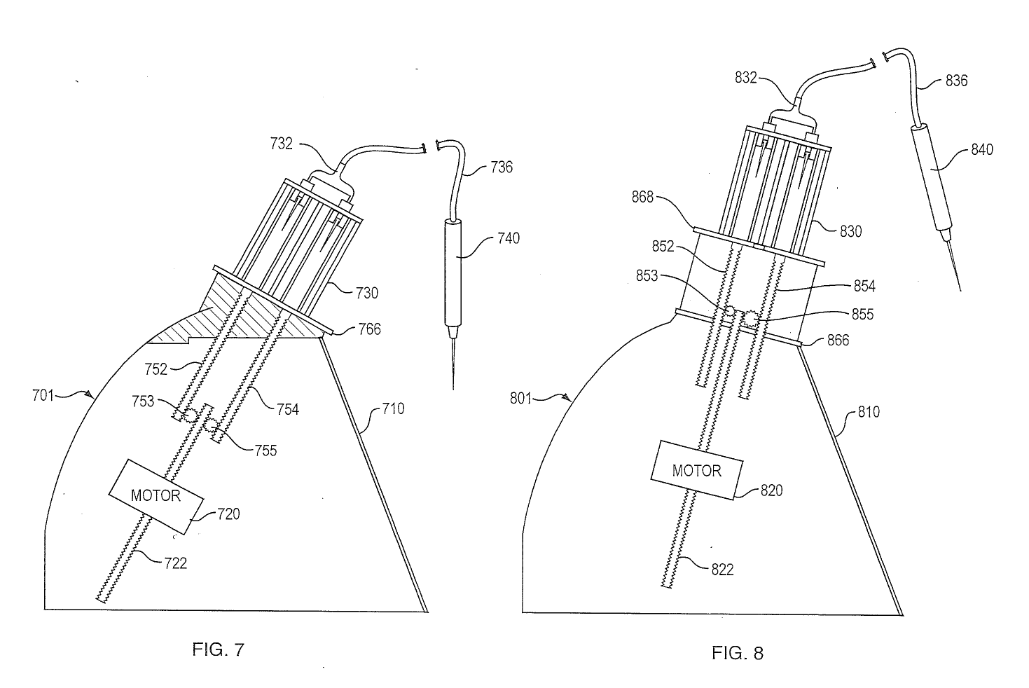

Referring now to FIGS. 7 and 8, these embodiments of the invention provide a differential gear mechanism for moving two cartridge plunges at different rates to therefore meter different and selected rates or volumes of the two liquids. This is achieved using a single plunger drive unit 701 or 801, such as those disclosed in U.S. Pat. No. 6,132,414 or 7,740,612 or 7,618,409 or 7,449,008, or the units sold by Milestone Scientific and known by the trademarks CompuDent®, THE WAND®, WAND-PLUS® and STA SINGLE TOOTH ANESTHESIA SYSTEM® Instruments.

The invention of FIGS. 7 and 8 each comprises a drive unit 701 or 801 with a motor 720 and 820 for linearly moving respective motor plungers 722 and 822, which also have rack teeth on their front and rear edges that mesh with pinion gears 753 and 755 in FIGS. 7 and 853 and 855 in FIG. 8. These pinion gears are mounted for rotation inside the respective drive unit housings 710 and 810, and mesh with rack teeth on the respective cartridge plungers 752, 754 in FIG. 7 and 852, 854 in FIG. 8. The internal gear mechanisms result in a differential rate/volume of administration for multi-plungers of a multi-cartridge housing adaptor 760 and 860 in conjunction to the use of a novel multi-cartridge holder 730 and 830 with the existing CompuDent®, THE WAND®, WAND-PLUS® and STA SINGLE TOOTH ANESTHESIA SYSTEM® Instruments.

In FIG. 7 the adaptor 760 is fixed as a top part of the unit housing 710 and in the embodiment of FIG. 8, the adaptor 860 is removable and can be attached to the housing 810 by a latch 866 on one side and to the holder 830 by a latch 868 on the other side. In the embodiment of FIG. 7, only one latch 766 for connecting the adaptor to the holder 730 is needed.

The gear driven mechanism for the multi-cartridge housing adaptor accepts two standard cartridge holders independently of each other and joined by a tubing “Y” connector. The system can also be used as single cartridge version as well or with a single dual-cartridge holder of the type shown in FIG. 2. The design discloses the use of this housing adaptor in conjunction with a modified CompuDent®, THE WAND®, WAND-PLUS® and STA SINGLE TOOTH ANESTHESIA SYSTEM® instrument or variety thereof.

The gear mechanism produces a differential rate of travel of more than one plunger resulting in a differential amount and/or rate of drug administration produced to multi-plungers of a Multi-Cartridge ADAPTOR for a Computer-Controlled Drug Delivery (MCD) Injection System instrument composed of two separate designs. The first design is a gear driven mechanism and the like for a multi-plunger adaptor that can be fitted to a standard single plunger driven drive unit. The standard C-CLAD drive unit single plunger would engage a Gear Mechanism housed in the Multi-Plunger Adapator and in turn will simultaneously move two independent cartridge plungers at a different rate and/or volume during the movement of the plungers when attached to a standard C-CLAD drive unit. Once attached the Multi-Plunger Adaptor is designed to allow two separate plungers to be translated at different rates during movement of the single drive plunger of the said drive unit. The Multi-Plunger-Adaptor of FIG. 7 will accept a disposable or re-usable “Dual Cartridge Holder”.

The invention is not limited to a dual cartridge system. It is understood that more than two cartridges may be used with a cartridge holder of the invention for accepting more than two standard or custom-made cartridges, and that two or more separate sources of drug or other liquid can be supplied to a single output source using the invention.

In FIG. 8 a standard C-CLAD instrument system is modified from a single plunger instrument, re-designed to accept a DUAL-PLUNGER Adaptor with Internal Gear Mechanism and the like in which the dual plungers are independent from one another and in contact with a single motor of the drive unit. The different Gear diameters would produce a different rate/volume of movement. A modified housing adaptor will replace the existing housing cartridge socket of the STA SINGLE TOOTH ANESTHESIA SYSTEM® drive unit and/or other instrument. A Dual-Cartridge Holder can attach to the modified multi-cartridge drive unit adaptor by numerous means, including, but not limited to, sliding, rotating, lock-n-key, etc. The Dual Cartridge Holder will deliver units of the two separate sources of drugs via a connector mixing element attached to a micro-tubing and handpiece/needle assembly.

It is understood that a differential rate of travel of multiple plungers can be produced by a different diameter of the Gear Mechanism contacting each plunger. The design is not limited to Gear Diameter and could also produce a differential rate of travel by varying the physical dimension of the diameter of the plunger and any other means of differential gear mechanisms. It is conceivable that multiple gears could be used to produce a rate/volume differential of multiple plungers used to express medications from more then one cartridge container. The invention is further not limited to the 1.8 ml glass dental cartridge. Others fluid volumes, sizes and configurations are anticipated by this disclosure and the design herein discussed.

While specific embodiments of the invention have been shown and described in detail to illustrate the application of the principles of the invention, it will be understood that the invention may be embodied otherwise without departing from such principles.

What is claimed is:

1. An apparatus for combining together and for injecting into a subject, a mixture of a liquid drug and at least one other liquid in desired proportions, the liquid drug and the at least one other liquid each being provided in respective sealed cartridges (114, 116), each cartridge having a cartridge cylinder (113) for containing a quantity of liquid with a sealed discharge end (115) and an opposite open end (117), a cartridge piston (119) in the cartridge cylinder near the opposite open end when the cartridge is full, the piston being pushable toward the discharge end for discharging liquid when the discharge end is punctured, the apparatus comprising:
a drive unit housing (110);
a motor (120) mounted in the drive unit housing;
a motor plunger (122) engaged to the motor for lineal movement with respect to the drive unit housing when the motor is activated;
a computer (124) connected to the motor and programmed with a program for activating the motor to move the motor plunger at selected times and at selected speeds;
a multiple cartridge holder (130) connected to the drive unit housing and adapted to receive a plurality of the cartridges with one of the cartridges containing the liquid drug and at least one other of the cartridges containing the other liquid;
a mixing chamber (132) operatively connected to the multiple cartridge holder (130) and having a plurality of inlets (134) each connectable to the discharge end of one of the cartridges received in the multiple cartridge holder for unsealing each respective discharge end for receiving liquid from each cartridge, the mixing chamber mixing liquids from the cartridges together and having an outlet (135) for discharging a mixture of the liquids;
a length of micro-tubing (136) having one end connected to the outlet of the mixing chamber and an opposite end;
a handpiece assembly (140) connected to the opposite end of the micro-tubing for receiving the mixture of liquid, the handpiece assembly having a subcutaneous injection needle (142) for injecting the mixture of liquids into a subject;
a plurality of cartridge plungers (152, 154) mounted for movement with respect to the drive unit housing (110) and into respective opposite open ends of cartridges (114, 116) received by the multiple cartridge holder (130) for pushing the respective cartridge pistons (119) toward the respective discharge ends to discharge liquids from the cartridges into the mixing chamber;
a motion transmission (160) engaged between the motor plunger (122) and the cartridge plungers (152, 154) for moving the cartridge plungers into the respective opposite open ends of the cartridges when the computer (124) activates the motor (120) to move the motor plunger; and
a foot controller (102) connected to the computer (124) for activating the motor via the program for combining, proportioning, mixing and injecting of the mixture in a hands-free manner.

2. The apparatus according to claim 1, wherein the holder and mixing chamber are made as one unit and of transparent plastic.

3. The apparatus according to claim 1, wherein the motion transmission comprises a plate connected on one side to the motor plunger and on an opposite side to all of the cartridge plungers.

4. The apparatus according to claim 1, wherein the motion transmission comprises an adaptor (360) connected between the drive unit housing (310) over the motor plunger (322), and the multiple cartridge holder (330), a plate mounted for movement in the adapter and connected to the cartridge plungers (352, 354), the plate being engaged and moved by the motor plunger for moving the cartridge plungers (352, 354).

5. The apparatus according to claim 1, wherein the motion transmission comprises an adaptor (460) having an adapter housing (461) connected to the drive unit housing (410) over the motor plunger (422), a plate (462) connected between the motor plunger (422) and the cartridge plungers (452, 454), the cartridge holder (430) being connected to the adapter housing (461) and the cartridge plungers (452, 454) being movable through the adaptor housing (461) and into the open end of cartridges (414, 416) received in the cartridge holder (430).

6. The apparatus according to claim 1, wherein the mixing chamber (432) comprises a Y-shaped section of micro-tubing having two arms (434, 434) forming the inlets of the mixing chamber and a leg (435) forming the mixing chamber outlet, the arms and leg being connected together at a junction for receiving liquids from each of the cartridges (414, 416) received in the cartridge holder (430).

7. The apparatus according to claim 1, wherein the motion transmission comprises an adaptor (560) connected to the drive unit housing (510) over the motor plunger (522), and the multiple cartridge holder comprising a separated holder (530, 531) for receiving each cartridge, a plate (562) mounted for movement to the adapter and connected to the cartridge plungers (552, 554), the plate being engaged and moved by the motor plunger (522) for moving the cartridge plungers.

8. The apparatus according to claim 1, wherein the mixing chamber comprises a Y-shaped section (532) having two arms (534) forming the inlets of the mixing chamber and a leg (535) forming the mixing chamber outlet, the arms and leg being connected together at a junction for receiving liquids from each of the cartridges (514, 516) received in the cartridge holder (430), the motion transmission comprising an adaptor (560) connected to the drive unit housing (510) over the motor plunger (522), and the multiple cartridge holder comprising a separated holder (530, 531) for receiving each cartridge, a plate (562) mounted for movement to the adapter and connected to the cartridge plungers (552, 554), the plate being engaged and moved by the motor plunger (522) for moving the cartridge plungers.

9. The apparatus according to claim 1, wherein the motion transmission comprises an adaptor (760, 860) connected to the drive unit housing over the motor plunger, and a differential gear mechanism between the motor plunger and the cartridge plungers for moving cartridge plungers by different amounts when the motor plunger is moved.

10. The apparatus according to claim 1, wherein the multiple cartridge holder (130) is adapted to receive two of the cartridges, the apparatus including, in combination, first and second sealed cartridges (114, 116) each having a cartridge cylinder (113) for containing a quantity of liquid with a sealed discharge end (115) and an opposite open end (117), a cartridge piston (119) in each cartridge cylinder near the opposite open end when the cartridge is full, the piston being pushable toward the discharge end for discharging liquid when the discharge end is punctured, the liquid in the first cartridge being an analgesic and the liquid in the second cartridge being a buffering solution for the analgesic.

11. In a drive unit comprising: a drive unit housing (110); a motor (120) mounted in the drive unit housing; a motor plunger (122) engaged to the motor for lineal movement with respect to the drive unit housing when the motor is activated; a computer (124) connected to the motor and programmed with a program for activating the motor to move the motor plunger at selected times and at selected speeds; and a foot controller (102) connected to the computer (124) for activating the motor for combining, proportioning, mixing and injecting of the mixture in a hands-free manner; the improvement comprising:
a apparatus for combining together and for injecting into a subject, a mixture of a liquid drug and at least one other liquid in desired proportions, the liquid drug and the at least one other liquid each being provided in respective sealed cartridges (114, 116), each cartridge having a cartridge cylinder (113) for containing a quantity of liquid with a sealed discharge end (115) and an opposite open end (117), a cartridge piston (119) in the cartridge cylinder near the opposite open end when the cartridge is full, the piston being pushable toward the discharge end for discharging liquid when the discharge end is punctured, the apparatus further comprising:
a multiple cartridge holder (130) connected to the drive unit housing and adapted to receive a plurality of the cartridges with one of the cartridges containing the liquid drug and at least one other of the cartridges containing the other liquid;
a mixing chamber (132) operatively connected to the multiple cartridge holder (130) and having a plurality of inlets (134) each connectable to the discharge end of one of the cartridges received in the multiple cartridge holder for unsealing each respective discharge end for receiving liquid from each cartridge, the mixing chamber mixing liquids from the cartridges together and having an outlet (135) for discharging a mixture of the liquids;
a length of micro-tubing (136) having one end connected to the outlet of the mixing chamber and an opposite end;
a handpiece assembly (140) connected to the opposite end of the micro-tubing for receiving the mixture of liquid, the handpiece assembly having a subcutaneous injection needle (142) for injecting the mixture of liquids into a subject;
a plurality of cartridge plungers (152, 154) mounted for movement with respect to the drive unit housing (110) and into respective opposite open ends of cartridges (114, 116) received by the multiple cartridge holder (130) for pushing the respective cartridge pistons (119) toward the respective discharge ends to discharge liquids from the cartridges into the mixing chamber; and
a motion transmission (160) engaged between the motor plunger (122) and the cartridge plungers (152, 154) for moving the cartridge plungers into the respective opposite open ends of the cartridges when the computer (124) activates the motor (120) to move the motor plunger;
wherein the foot controller (102) connected to the computer (124) for activating the motor via the program facilitates combining, proportioning, mixing and injecting of the mixture in a hands-free manner.

12. The apparatus according to claim 11, wherein the holder and mixing chamber are make as one unit and of transparent plastic.

13. The apparatus according to claim 11, wherein the motion transmission comprises a plate connected on one side to the motor plunger and on an opposite side to the cartridge plungers.

14. The apparatus according to claim 11, wherein the motion transmission comprises an adaptor (360) connected between the drive unit housing (310) over the motor plunger (322), and the multiple cartridge holder (330), a plate mounted for movement in the adapter and connected to the cartridge plungers (352, 354), the plate being engaged and moved by the motor plunger for moving the cartridge plungers (352, 354).

15. The apparatus according to claim 11, wherein the motion transmission comprises an adaptor (460) having an adapter housing (461) connected to the drive unit housing (410) over the motor plunger (422), a plate (462) connected between the motor plunger (422) and the cartridge plungers (452, 454), the cartridge holder (430) being connected to the adapter housing (461) and the cartridge plungers (452, 454) being movable through the adaptor housing (461) and into the open end of cartridges (414, 416) received in the cartridge holder (430).

16. The apparatus according to claim 11, wherein the mixing chamber (432) comprises a Y-shaped section of micro-tubing having two arms (434, 434) forming the inlets of the mixing chamber and a leg (435) forming the mixing chamber outlet, the arms and leg being connected together at a junction for receiving liquids from each of the cartridges (414, 416) received in the cartridge holder (430).

17. The apparatus according to claim 11, wherein the motion transmission comprises an adaptor (560) connected to the drive unit housing (510) over the motor plunger (522), and the multiple cartridge holder comprising a separated holder (530, 531) for receiving each cartridge, a plate (562) mounted for movement to the adapter and connected to the cartridge plungers (552, 554), the plate being engages and moved by the motor plunger (522) for moving the cartridge plungers.

18. The apparatus according to claim 11, wherein the mixing chamber comprises a Y-shaped section (532) having two arms (534) forming the inlets of the mixing chamber and a leg (535) forming the mixing chamber outlet, the arms and leg being connected together at a junction for receiving liquids from each of the cartridges (514, 516) received in the cartridge holder (430), the motion transmission comprising an adaptor (560) connected to the drive unit housing (510) over the motor plunger (522), and the multiple cartridge holder comprising a separated holder (530, 531) for receiving each cartridge, a plate (562) mounted for movement to the adapter and connected to the cartridge plungers (552, 554), the plate being engaged and moved by the motor plunger (522) for moving the cartridge plungers.

19. The apparatus according to claim 11, wherein the motion transmission comprises an adaptor (760, 860) connected to the drive unit housing over the motor plunger, and a differential gear mechanism between the motor plunger and the cartridge plungers for moving cartridge plungers by different amount when the motor plunger is moved.

20. The apparatus according to claim 11, wherein the multiple cartridge holder (130) is adapted to receive two of the cartridges, the apparatus including, in combination, first and second sealed cartridges (114, 116) each having a cartridge cylinder (113) for containing a quantity of liquid with a sealed discharge end (115) and an opposite open end (117), a cartridge piston (119) in each cartridge cylinder near the opposite open end when the cartridge is full, the piston being pushable toward the discharge end for discharging liquid when the discharge end is punctured, the liquid in the first cartridge being an analgesic and the liquid in the second cartridge being a buffering solution for the analgesic.

21. A method for combining together and for injecting into a subject, a mixture of a liquid drug and at least one other liquid in desired proportions, the liquid drug and the at least one other liquid each being provided in respective sealed cartridges (114, 116), each cartridge having a cartridge cylinder (113) for containing a quantity of liquid with a sealed discharge end (115) and an opposite open end (117), a cartridge piston (119) in the cartridge cylinder near the opposite open end when the cartridge is full, the piston being pushable toward the discharge end for discharging liquid when the discharge end is punctured, the method comprising:
providing a drive unit housing (110);
providing a motor (120) mounted in the drive unit housing;
providing a motor plunger (122) engaged to the motor for lineal movement with respect to the drive unit housing when the motor is activated;
providing a computer (124) connected to the motor and programmed with a program for activating the motor to move the motor plunger at selected times and at selected speeds;
providing a multiple cartridge holder (130) connected to the drive unit housing and adapted to receive a plurality of the cartridges with one of the cartridges containing the liquid drug and at least one other of the cartridges containing the other liquid;
providing a mixing chamber (132) operatively connected to the multiple cartridge holder (130) and having a plurality of inlets (134) each connectable to the discharge end of one of the cartridges received in the multiple cartridge holder for unsealing each respective discharge end for receiving liquid from each cartridge, the mixing chamber mixing liquids from the cartridges together and having an outlet (135) for discharging a mixture of the liquids;
providing a length of micro-tubing (136) having one end connected to the outlet of the mixing chamber and an opposite end;
providing a handpiece assembly (140) connected to the opposite end of the micro-tubing for receiving the mixture of liquid, the handpiece assembly having a subcutaneous injection needle (142) for injecting the mixture of liquids into a subject;
providing a plurality of cartridge plungers (152, 154) mounted for movement with respect to the drive unit housing (110) and into respective opposite open ends of cartridges (114, 116) received by the multiple cartridge holder (130) for pushing the respective cartridge pistons (119) toward the respective discharge ends to discharge liquids from the cartridges into the mixing chamber;
providing a foot controller (102) connected to the computer (124) for activating the motor via the program;
using a motion transmission (160) engaged between the motor plunger (122) and the cartridge plungers (152, 154) to move the cartridge plungers into the respective opposite open ends of the cartridges when the computer (124) activates the motor (120) to move the motor plunger; and
depressing the foot controller (102) for activating the motor via the program to combine, proportion, mix and inject the mixture in a hands-free manner.
